(19) United States
(12) Patent Application Publication
(10) Pub. No.: US 2011/0015736 A1
(43) Pub. Date: Jan. 20, 2011

(54) Temporary Implantable Medical Device

(76) Inventors: Hamid Sharim, Ramat Gan (IL); Shmuel Beck, Ramat Gan (IL)

Correspondence Address: The Law Office of Michael E. Kondoudis, 888 16th Street, N.W., Suite 800, Washington, DC, 20006, US

(21) Appl. No.: 12/665,067

(22) PCT Filed: Jun. 18, 2008
(86) PCT No.: PCT/IL08/00832

§ 371 Date: Sep. 12, 2010

Related U.S. Application Data
(60) Provisional application No. 60/929,208, filed on Jun. 18, 2007.

(51) Int. Cl. A61F 2/02; A61M 31/00

(52) U.S. Cl. 623/11.11; 604/500

(57) ABSTRACT

A temporarily implantable medical device is disclosed. Said device is implantable in a predetermined body portion to be treated; wherein said device comprises (a) a reversibly implantable elongated continuous strip of a predetermined shape having a biocompatible surface; said strip having a distal end within the body of a patient and a proximal end located adjacent to the subcutaneous tissue of said body portion to be treated; and, (b) a cannula having a least one exposed portion located at said subcutaneous tissue of said body portion to be treated, accommodating at least a portion of said proximal end of said strip; said cannula comprises sealing means adapted to seal said cannula.

### CROSS-REFERENCE TO RELATED APPLICATIONS

This application is the National Stage of International Application No. PCT/IL2008/000832, filed Jun. 18, 2008, which claims the benefit of U.S. Provisional Application No. 60/929,208, filed Jun. 18, 2007.

### FIELD OF THE INVENTION

The present invention relates to tissue molding and/or re-molding and/or construction and/or reconstruction by using the temporarily implantable medical device provided by the present invention.

### BACKGROUND OF THE INVENTION

The present invention relates to tissue molding and/or re-molding in general. One specific example is the use of the implantable medical device provided by the present invention in spinal procedures and, more specifically, to prevent postoperative adhesions forming on vertebrae and/or tendons and/or nerve roots and surrounding tissues. It is emphasized, however, that the device can be used in different fields such as laminectomy, laminoplasty, scaffolding, filler design, plastic surgery, sinus lifting, sinus separation, craniotomy, carpal tunnel surgery, or any operation on a ligament, a tendon, a sinew, a string, or a cord.

After a surgical procedure, adhesions are commonly formed between an organ, surrounding connective tissue and bone. Following surgical trauma, connective tissue surrounding the organ proliferates to form a fibrous mass that binds the organ to neighbouring organs, viscera, muscle, or bone. Depending on the type of surgery and the location of the incision, the adhesions may produce negligible discomfort or severe pain originating from the root of the nerve. However, adhesion formation can significantly complicate subsequent surgical procedures at the same or adjacent sites. Repeat surgical procedures are fairly frequent in the back, heart, abdomen and cranium. The presence of post-operative adhesions from a prior surgery complicates the second surgery because the contacts between the target organ and the neighbouring bone and connective tissue must be carefully dissected away before the surgeon can initiate the corrective surgical procedure. The surgeon risks damaging the target organ during the dissection and the time required for the dissection procedures adds to the total time that the patient is under general anaesthesia.

Scar tissue formation after laminectomies and laminotomies for disk excision or decompressive laminectomy for spinal stenosis present both surgeon and patient with an additional post-operative concern. Laminectomies and laminotomies frequently remove bone tissue and leave the dura exposed. Post-laminectomy scar tissue, also termed epidural fibrosis, is primarily formed from fibrous connective tissue and develops in the post-operative hematoma that forms between the paraspinous muscles and the dura. The dura is relatively thin and can easily be injured during surgery. In particular, the dura is susceptible to damage during revision surgery when scar tissue adheres to the dura making it difficult for the surgeon to perform an adequate neurolysis. Thus, a method is needed for protecting the dura from scar tissue adhesion.

At the present time, methods to minimize the amount of scar tissue include the use of autogenous fat grafts, gelatin foams or sponges, or microfibullary collagen as an interposing protective layer between the spinal dura and the adjacent viscera. Other biological substances and chemical compounds that have been tested experimentally for their usefulness in animals include bone grafts, microfibrillar collagen, elastase, polyethylene, mylar, dacron, teflon and methylmethacrylate.

Autogenous fat grafts have been used following laminectomies as early as 1964. The fat is placed over the exposed dura after removal of the lamina or a portion of the lamina. The fat provides a protective barrier for the dura, and may limit scar formation between the muscle and the dural tissue. However, it is known that fat grafts frequently adhere to the dura. These adhesions complicate revision surgery because they require tedious dissection by the orthopaedic surgeon or neurosurgeon. Fat grafts are preferably harvested from a site close to the surgical incision, such as the subdermal areolar tissue bed. However, unless the patients are overweight, fat harvesting from nearby locations is not always possible, particularly in multiple laminectomy procedures. Further, fat harvesting may require a second incision. The incisions at the secondary locations may sometimes lead to complications such as hematoma formation or dimpling in the skin.

Other substances are used where fat grafts are not possible or desired. Gelatin foam (such as Gelfoam® sponge, supplied by Upjohn Company Inc., Kalamazoo, Mich.), or polylactic acid (PLA) is a useful substitute for autogenous fat grafts. This material is also placed over the dura to reduce scar formation. There is some controversy concerning the preference of gelatin foams or sponges versus fat; however, neither is optimal. Like fat, gelatin foams or sponges may move out of position following surgery. Furthermore, while fat and gelatin foams may form a barrier between the visceral tissue and the dura, there is a propensity for both fat and gelatin foam or sponge to adhere to the dura. Neither fat nor gelatin foam provides adequate physical protection to the cauda equina.

A mechanical barrier that would provide support to the spinal dura as well as reduce scar formation is needed. U.S. Pat. No. 4,013,078 to Feild discloses a device for preventing adhesions between the patient's dura and spinal nerves and other anatomic structures following spinal surgery. The device includes a conduit sheath of teflon or silicone that is positioned in close proximity to the nerve root. Like the previous protective overlay substances, such a device is invasive to the neuroforamen and anchors directly to the dura. This in turn would promote adhesions between the dura and the protecting device creating unnecessary complications for revision surgery.

In order to minimize the surgical time for dissection, minimize nerve injury, and minimize dural tears, a spinal cord protection device should be simple to insert, non-invasive to the dura, and maintain a distance from the neural tissues. Preferably, anchoring means should contact bone instead of tissue prone to scar formation to minimize post-operative epidural fibrosis. Finally, the optimal mechanical device is readily contoured to provide a customized mechanical barrier to prevent dural or nerve root injury. Preferably, the device is adaptable in design to accommodate other surgical devices used in back surgery. Such a device is provided in the detailed description of this invention.

Adhesions also form between the heart and the anterior thoracic skeleton following cardiac surgery. In particular, adhesions form between the posterior surface of the sternum and the anterior surfaces of the heart. Repeat open heart surgeries are complicated by adhesion formation because the scar tissue must be dissected away before the sternum can be cut lengthwise and before the anterior thoracic skeleton can be retracted to expose the heart. For example, it is estimated that there are at least 250,000 coronary artery bypass graft surgeries done each year in the United States. Approximately 20% of these surgeries are revision surgeries. Adhesions form between the greater vessels of the heart and the posterior surface of the sternum. The adhesions make the separation of the pericardium from the sternum difficult and thus create severe complications during revision surgeries. It is estimated that 2 to 4% of the revision surgeries end in fatality as a result of adhesion-induced complications. Therefore, there is a need for a device that minimizes adhesion formation. The present device fulfills this need. Moreover, the device is simple to insert, easy to remove, and prevents the formation of adhesions between the heart and the posterior surface of the sternum.

U.S. Pat. No. 6,454,767 ('767) discloses a spinal protection device minimizing the formation of post-operative adhesions. The protection device constitutes a fenestrated shield, and may be positioned such that contact between the shield and the spinal dura is substantially avoided.

A method of performing spinal surgery comprising the steps of: performing a bony dissection on at least a portion of one vertebra to expose the spinal dura; and positioning a shield to cover said exposed spinal dura, wherein said shield covers at least a portion of one vertebra having said bony dissection, wherein said shield is spaced apart from the spinal dura.

The prior art analysis indicates a lack of ability of the spinal shield to be withdrawn after cicatrizing the area of the surgical operation. Spinal shields made of biodegradable materials are known. It must be admitted, however, that withdrawing the spinal shield after cicatrizing the operation area is preferable because no foreign object remains in the operation area. Thus, providing a temporarily implantable medical device adapted to prevent surrounding tissues at an area of a surgical operation from mutual adhesion in a period of cicatrisation is an unmet long-felt need.

As mentioned above the present invention is not limited to spinal procedures, but rather answers a general need in molding, re-molding, construction and re-construction of tissues.

### SUMMARY OF THE INVENTION

It is hence one object of the invention to disclose a reversibly implantable medical device adapted for molding and/or re-molding and/or constructing and/or reconstructing tissues; said device is implantable in a predetermined body portion to be treated; wherein said device comprises:
a. a reversibly implantable elongated continuous strip of a predetermined shape having a biocompatible surface; said strip having a distal end within the body of a patient and a proximal end located adjacent to the subcutaneous tissue of said body portion to be treated; and,
b. a cannula having a least one exposed portion located at said subcutaneous tissue of said body portion to be treated, accommodating at least a portion of said proximal end of said strip; said cannula comprises sealing means adapted to seal said cannula.

It is hence another object of the present invention to disclose the reversibly implantable medical device as defined above, adapted to prevent tissue adhesion, especially post-operative tissue adhesions.

It is hence another object of the present invention to disclose the reversibly implantable medical device as defined above, especially adapted for medical procedures selected from a group consisting of laminectomy, laminoplasty, scaffolding, filler design, plastic surgery, tissue reconstruction, tissue construction, sinus lifting, sinus separation, craniotomy, carpal tunnel surgery, and any operation on a ligament, a tendon, a sinew, a string, or a cord.

It is hence another object of the present invention to disclose the reversibly implantable medical device as defined above, wherein said strip is adapted to form either a 2D or 3D spatial barrier.

It is hence another object of the present invention to disclose the reversibly implantable medical device as defined above, wherein the shape of said barrier is selected from a group consisting of regular or irregular spirally wound, zigzag, lumen, pipe, crescent, linear, bagel-like (annular) cross section, star-like cross section, otherwise a planar or proximally planar shaped barrier of any size, shape or type, 3D configuration, and a multi-dimensional configuration comprising a plurality of regular or irregular two or more 2D planes and/or 3D spatial members interlinked together to form a continuous member suitable to at least partially fill the interstices between tissues and/or organs within the body.

It is hence another object of the present invention to disclose the reversibly implantable medical device as defined above, wherein said tissue and/or organs are selected from a group consisting of a ligament, a tendon, a sinew, a string, peripheral nerve and surrounding tissues, a cord, and surrounding tissues thereof.

It is hence another object of the present invention to disclose the reversibly implantable medical device as defined above, wherein said cannula is at least temporarily affixed to patient's subcutis and/or facia.

It is hence another object of the present invention to disclose the reversibly implantable medical device as defined above, wherein said strip is coated and/or immersed and/or coupled and/or doped and/or covered and/or contained by means selected from a group consisting of anti-adhesive agents, medicaments, sustained released drugs, radio-opaque agents, bio-markers, biodegradable polymer, fillers, edema preventing members, biocides, shape memory materials, collagen enzymes, anti-coagulating agents, proadhesion modifiers such as talcum powder, Si containing agents.

It is hence another object of the present invention to disclose the reversibly implantable medical device as defined above, wherein said cannula having a distal end inserted into said body portion to be treated and a proximal end located adjacent to the subcutaneous tissue of said body portion to be treated; further wherein the shape of said distal end is concave and/or flaring.

It is hence another object of the present invention to disclose the reversibly implantable medical device as defined above, wherein said strip and/or cannula are flexible or rigid and are composed of materials selected from a group consisting of metals such as stainless steel (SS), annealed 316 SS, Nitinol (NiTi); silica, polyurethane, polyamides, silk mesh, biodegradable polymer such as PLA (polylactic acid), PGA (polyglycolic acid).

It is hence another object of the present invention to disclose the reversibly implantable medical device as defined above, wherein said strip having thickness of about 50 microns to about 500 microns, preferably about 100 microns to about 200 microns; length of about 3 centimetres to about 3 meters; width of about 1 millimetre to about 3 millimetres, preferably about 1.5 millimetres.

It is hence another object of the present invention to disclose a method for treating a predetermined body portion by molding and/or re-molding and/or constructing and/or reconstructing tissues. The method comprises steps selected inter alia from:
a. reversibly implanting an elongated continuous strip of a predetermined shape, having a biocompatible surface, within said body portion to be treated by inserting said strip to said body portion to be treated;
b. shaping said strip within said body portion to be treated according to said predetermined shape; and,
c. providing at least a portion of said strip adjacent to the subcutaneous tissue of said body portion to be treated.

It is hence another object of the present invention to disclose the method as defined above, additionally comprising the final steps of:
a. reversibly locating at least a portion of a cannula at said subcutaneous tissue of said body portion to be treated and optionally at least temporarily fixating the same;
b. withdrawing said strip out of said body via said cannula.

It is hence another object of the present invention to disclose the method as defined above, for preventing tissue adhesion in a predetermined body portion to be treated, especially post-operative tissue adhesions; preferably comprising cicatrizing tissue.

It is hence another object of the present invention to disclose the method as defined above, useful for surgical operation selected from a group consisting of laminectomy, laminoplasty, scaffolding, filler design, plastic surgery, tissue reconstruction, tissue construction, sinus lifting, sinus separation, craniotomy, carpal tunnel surgery, and any operation on a ligament, a tendon, a sinew, a string, or a cord.

It is hence another object of the present invention to disclose a minimally invasive method as defined above.

It is hence another object of the present invention to disclose the method as defined above, additionally comprising step of coating and/or immersing and/or coupling and/or doping and/or covering and/or containing said strip by means selected from a group consisting of anti-adhesive agents, non-adhesive agents, medicaments, sustained released drugs, radio opaque agents, bio-markers, biodegradable polymers, fillers, edema preventing members, biocides, anti-coagulating agents.

It is hence another object of the present invention to disclose a reversibly implantable medical device adapted for molding and/or re-molding and/or constructing and/or reconstructing tissues, said device being implantable in a predetermined body portion to be treated. Said device comprises:
a. a reversibly implantable elongated continuous strip of a predetermined shape having a biocompatible surface; said strip having a distal end within the body of a patient and a proximal end located adjacent to the subcutaneous tissue of said body portion to be treated; and,
b. a cannula having a least one exposed portion located at said subcutaneous tissue of said body portion to be treated, accommodating at least a portion of said proximal end of said strip; said cannula comprising sealing means adapted to seal said cannula; and,
c. membrane interlinks or couplings for at least two portions of said strip in said distal end within said body.

It is hence another object of the present invention to disclose the reversibly implantable medical device as defined above, wherein said membrane provides backing support for said strip.

It is hence another object of the present invention to disclose the reversibly implantable medical device as defined above, adapted to prevent tissue adhesion, especially post-operative tissue adhesions.

It is hence another object of the present invention to disclose the reversibly implantable medical device as defined above, especially adapted for medical procedures selected from a group consisting of laminectomy, laminoplasty, scaffolding, filler design, plastic surgery, tissue reconstruction, tissue construction, sinus lifting, sinus separation, craniotomy, carpal tunnel surgery, and any operation on a ligament, a tendon, a sinew, a string, or a cord.

It is hence another object of the present invention to disclose the reversibly implantable medical device as defined above, wherein said strip is adapted to form either a 2D or 3D spatial barrier.

It is hence another object of the present invention to disclose the reversibly implantable medical device as defined above, wherein the shape of said barrier is selected from a group consisting of regular or irregular spirally wound, zigzag, lumen, pipe, crescent, linear, bagel-like (annular) cross section, star-like cross section, otherwise a planar or proximally planar shaped barrier of any size, shape or type, 3D configuration, and a multi-dimensional configuration comprising a plurality of regular or irregular two or more 2D planes and/or 3D spatial members interlinked together to form a continuous member suitable to at least partially fill the interstices between tissues and/or organs within the body.

It is hence another object of the present invention to disclose the reversibly implantable medical device as defined above, wherein said tissue and/or organs are selected from a group consisting of a ligament, a tendon, a sinew, a string, peripheral nerve and surrounding tissues, a cord, and surrounding tissues thereof.

It is hence another object of the present invention to disclose the reversibly implantable medical device as defined above, wherein said cannula is at least temporarily affixed to patient's subcutis and/or facia.

It is hence another object of the present invention to disclose the reversibly implantable medical device as defined above, wherein said strip is coated and/or immersed and/or coupled and/or doped and/or covered and/or contained by means selected from a group consisting of anti-adhesive agents, medicaments, sustained released drugs, radio opaque agents, bio-markers, biodegradable polymers, fillers, edema preventing members, biocides, shape memory materials, collagen enzymes, anti coagulating agents, proadhesion modifiers such as talcum powder, Si containing agents.

It is hence another object of the present invention to disclose the reversibly implantable medical device as defined above, wherein said cannula having a distal end inserted into said body portion to be treated and a proximal end located adjacent to the subcutaneous tissue of said body portion to be treated; further wherein the shape of said distal end is concave and/or flared.

It is hence another object of the present invention to disclose the reversibly implantable medical device as defined above, wherein said strip and/or cannula and/or said membrane are flexible or rigid and are composed of materials selected from a group consisting of metals such as stainless steel (SS), annealed 316 SS, Nitinol (NiTi), silica, polyurethane, polyamides, silk mesh, biodegradable polymers such as PLA (polylactic acid), PGA (polyglycolic acid).

It is hence another object of the present invention to disclose the reversibly implantable medical device as defined above, wherein said membrane is produced by means of gluing or deeping, immersing, laser cutting, photoetching, moulding or thermoshaping.

It is hence another object of the present invention to disclose the reversibly implantable medical device as defined above, wherein said strip having thickness of about 50 microns to about 500 microns, preferably about 100 microns to about 200 microns; length of about 3 centimetres to about 3 meters; width of about 1 millimetre to about 3 millimetres, preferably about 1.5 millimetres.

It is hence another object of the present invention to disclose a method for treating a predetermined body portion by molding and/or re-molding and/or constructing and/or reconstructing tissues. The method comprises steps selected inter alia from
a. providing an elongated continuous strip of a predetermined shape, having a biocompatible surface;
b. interlinking or coupling several portions of said elongated continuous strip via a membrane;
c. reversibly implanting said elongated continuous strip within said body portion to be treated by inserting said strip to said body portion to be treated;
d. shaping said strip within said body portion to be treated according to said predetermined shape; and,
e. providing at least a portion of said strip adjacent to the subcutaneous tissue of said body portion to be treated.

It is hence another object of the present invention to disclose the method as defined above, additionally comprising the final steps of:
a. reversibly locating at least a portion of a cannula at said subcutaneous tissue of said body portion to be treated and optionally at least temporarily fixating the same;
b. withdrawing said strip out of body via said cannula.

It is hence another object of the present invention to disclose the method as defined above, for preventing tissue adhesion in a predetermined body portion to be treated, especially post-operative tissue adhesions; preferably with cicatrizing tissue.

It is hence another object of the present invention to disclose the method as defined above, useful for surgical operation selected from a group consisting of laminectomy, laminoplasty, scaffolding, filler design, plastic surgery, tissue reconstruction, tissue construction, sinus lifting, sinus separation, craniotomy, carpal tunnel surgery, and any operation on a ligament, a tendon, a sinew, a string, or a cord.

It is another object of the present invention to disclose a minimally invasive method as defined above.

It is lastly an another object of the present invention to disclose the method as defined above, additionally comprising step of coating and/or immersing and/or coupling and/or doping and/or covering and/or containing said strip by means selected from a group consisting of anti-adhesive agents, non-adhesive agents, medicaments, sustained released drugs, radio-opaque agents, bio-markers, biodegradable polymers, fillers, edema preventing members, biocides, anti-coagulating agents.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings, in which

FIG. 1a is a schematic isometric view of the spiral implantable medical device;

FIG. 1b is a schematic isometric view of the zigzag implantable medical device;

FIGS. 1c-1d is a schematic isometric view of another embodiment of the implantable medical device;

FIG. 2a is a schematic diagram of scar formation after a wide laminectomy;

FIG. 2b is a top view of the zigzag laminar member deployed in the operation area;

FIG. 2c is a front view of the zigzag laminar member deployed in the operation area;

FIG. 2d is a view of the zigzag laminar member with the fixating tubular member;

FIG. 3a is a schematic diagram of the human carpus;

FIG. 3b is a schematic diagram of the medial nerve and the transverse ligament before the carpal tunnel surgical operation;

FIG. 3c is a schematic diagram of the medial nerve and the transverse ligament after the carpal tunnel surgical operation;

FIG. 3d is a cross-sectional diagram of the carpus with the implanted spiral device; and

FIG. 3e is a enlarged cross-sectional diagram of the carpus with the implanted spiral device;

FIGS. 4a, 4b, 5a, 5b, 6a, 6b, 7a and 7b schematically illustrate the use of the implantable device provided by the present invention in different body areas (the shoulders, FIGS. 4a and 4b; in the arm, FIGS. 5a and 5b; in the hip or thigh area, FIGS. 6a and 6b; in the face, FIGS. 7a and 7b).

### DETAILED DESCRIPTION OF THE INVENTION

The following description is provided in order to enable any person skilled in the art to make use of said invention and sets forth the best modes contemplated by the inventor of carrying out this invention. Various modifications, however, will be apparent to those skilled in the art, since the generic principles of the present invention have been defined specifically to provide an implantable medical device and a method of using the same, and therefore the invention is not limited by that which is illustrated in the figures and described in the specification, but only as indicated in the accompanying claims, with the proper scope determined only by the broadest interpretation of said claims.

The term ‘laminectomy’ hereinafter refers to a spine operation to remove the portion of the vertebral bone called the lamina. There are many varieties of laminectomy. In the most minimal form small skin incisions are made, back muscles are pushed aside rather than cut, and the parts of the vertebra adjacent to the lamina are left intact. The traditional form of laminectomy (conventional laminectomy) excises much more than just the lamina: the entire posterior backbone is removed, along with overlying ligaments and muscles. The usual recovery period is very different depending on which type of laminectomy has been performed: days in the minimal procedure, and weeks to months with conventional open surgery.

The lamina is a posterior arch of the vertebral bone lying between the spinous process, which juts out in the midline, and the more lateral of each vertebra. The pair of lamina, along with the spinous process, make up the posterior wall of the bony spinal canal. Although the literal meaning of laminectomy is excision of the lamina, the operation called conventional laminectomy, which is a standard spinal procedure in neurosurgery and orthopedics, removes the lamina, spinous process and overlying connective tissues and ligaments, cutting through the muscles that overlie these structures. Minimal surgery laminectomy is a tissue preserving surgery that leaves the muscles intact, spares the spinal process and takes only one or both lamina. Laminotomy is removal of a mid-portion of one lamina and may be done either with a conventional open technique, or in a minimal fashion with the use of tubular retractors and endoscopes.

A lamina is rarely, if ever, removed because it itself is diseased. Instead, removal is done to: (1) break the continuity of the rigid ring of the spinal canal to allow the soft tissues within the canal to expand (decompression), or (2) as one step in changing the contour of the vertebral column, or (3) in order to allow the surgeon access to deeper tissues inside the spinal canal. Laminectomy is also the name of a spinal operation that conventionally includes the removal of one or both lamina as well as other posterior supporting structures of the vertebral column, including ligaments and additional bone.

The term “about” refers hereinafter to a range of 25% below or above the referred value.

The terms ‘carpal tunnel syndrome’ (CTS) or median neuropathy at the wrist hereinafter refer to a medical condition in which the median nerve is compressed at the wrist, leading to pain, paresthesias, and muscle weakness in the forearm and hand. A form of compressive neuropathy, CTS is more common in women than it is in men and has a peak incidence around age 42, though it can occur at any age. The lifetime risk for CTS is around 10% of the adult population.

The term ‘ligament’ hereinafter refers to a tough fibrous band of tissue connecting the articular extremities of bones or supporting an organ in place.

The term ‘tendon’ or ‘sinew’ hereinafter refers to a tough band of fibrous connective tissue that usually connects muscle to bone and is capable of withstanding tension. Tendons are similar to ligaments except that ligaments join one bone to another.

The term “laminoplasty” hereinafter refers to a surgical procedure for treating spinal stenosis. The procedure is often used in patients with painfully restricted spinal canals in their necks. The procedure immediately relieves pressure by creating more space for the spinal cord and roots. The device that is provided by the present invention is used for the reconstruction of the lamina.

The present invention provides a reversibly implantable medical device to be use in different medical procedures such as prevention of tissue adhesion, especially post-operative tissue adhesions, laminectomy, laminoplasty, scaffolding, plastic surgeries, tissue reconstruction, tissue construction, sinus lifting, sinus separation, craniotomy, carpal tunnel surgery, and any operation on a ligament, a tendon, a sinew, a string, or a cord.

The reversibly implantable medical device is implantable in a predetermined body portion to be treated and comprises inter alia the following:
(a) a reversibly implantable elongated continuous strip of a predetermined shape having a biocompatible surface; said strip having a distal end within the body of a patient and a proximal end located adjacent to the subcutaneous tissue of said body portion to be treated; and,
(b) a cannula having a least one exposed portion located at said subcutaneous tissue of said body portion to be treated, accommodating at least a portion of said proximal end of said strip; said cannula comprises sealing means adapted to seal said cannula.

The present invention also provides a method of using the reversibly implantable medical device. The method comprises inter alia the following steps:
(a) reversibly implanting an elongated continuous strip of a predetermined shape, having a biocompatible surface, within said body portion to be treated by inserting said strip to said body portion to be treated;
(b) shaping said strip within said body portion to be treated according to said predetermined shape; and
(c) providing at least a portion of said strip adjacent to the subcutaneous tissue of said body portion to be treated.

The final steps are:
(d) reversibly locating at least a portion of a cannula at said subcutaneous tissue of said body portion to be treated and optionally at least temporarily fixating the same;
(e) withdrawing said strip out of body via said cannula.

It should be emphasized that the device and method as provided by the present invention can be used in different medical procedures such as:
(a) laminoplasty surgeries;
(b) plastic procedures—for tissue molding, tissue construction and re-construction, filling spaces or fragmentariness (e.g., cicatrix);
(c) scaffolding;
(d) craniotomy;
(e) carpal tunnel surgery, et cetera.

The following description is given as an example of the use of the above mentioned implantable medical device in spinal surgical procedures.

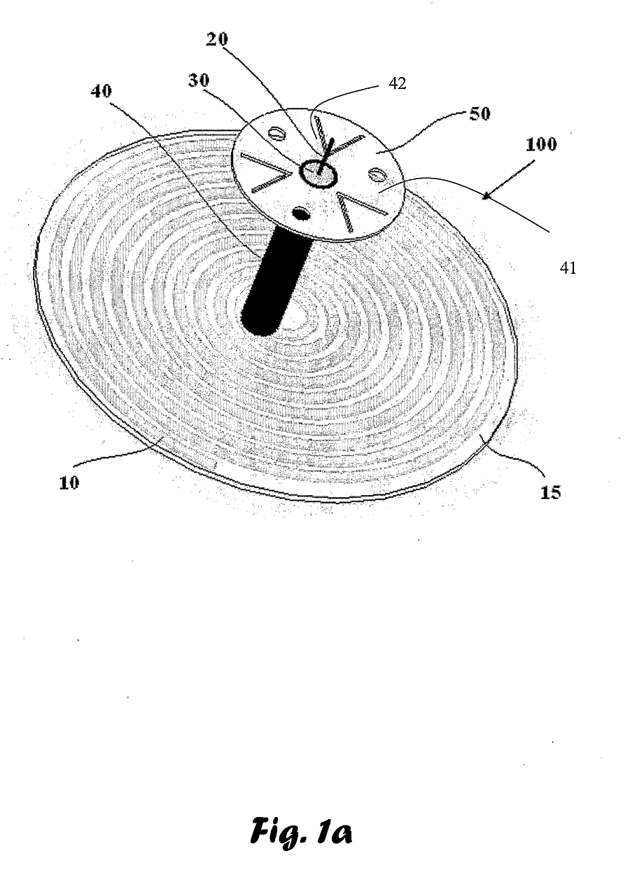

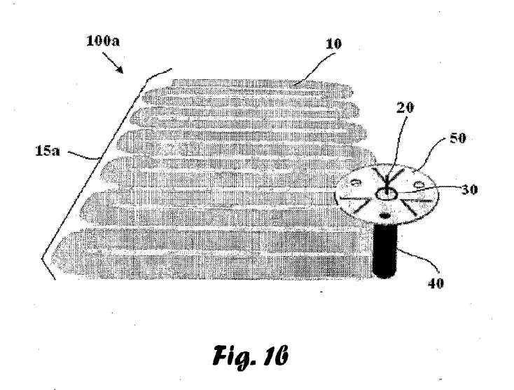

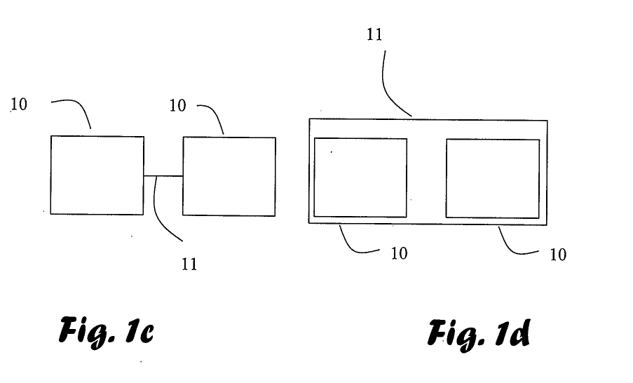

Reference is now made to FIG. 1a, showing an implantable medical device 100. The aforesaid device 100 comprises a linear member 10 releasably configured into a spiral laminar member 15, a hollow tubular member (cannula) 40 accommodating a proximal terminal of the linear member 10. A plate 50 is adapted for subcutaneous fixation for a period of cicatrisation. As seen in FIG. 1a, the aforesaid linear member has a core 20 and a biocompatible jacket 30. According to one of the embodiments of the current invention, the core 20 is provided with a knob (not shown) at the proximal end thereof. Mechanical properties of the linear member 10 enable the surgeon to deploy the spirally configured laminar member 15 in an area of a surgical operation (not shown) and withdraw it through the cannula 40 pulling it out. The laminar member 15 is adapted for unraveling into the linear member 10.

It should be pointed out that the tubular member (cannula) 40 in FIG. 1a is centralized. Said cannula 40 is not necessarily in the center, however, and it can be coupled to member 10 at any other location.

The tubular member (cannula) 40 can additionally comprise means (41) for fixating said cannula 40 by sewing or by tacks (42).

Reference is now made to FIG. 1b, presenting an implantable zigzag medical device 100a comprising a laminar member 15a made of the zigzag-configured linear member 10. Similar to FIG. 1a, the proximal terminal of the linear member is disposed into the cannula 40. The zigzag device is adapted to be disposed in the area of the surgical operation and withdrawn from that area by means of pulling out the linear member 10 through the cannula 40.

Reference is now made to FIGS. 1c and 1d illustrating other embodiments of the present invention. According to this embodiment, a reversibly implantable medical device implantable in a predetermined body portion to be treated is provided. The device comprises:
a. a reversibly implantable elongated continuous strip (10) of a predetermined shape having a biocompatible surface. The strip has a distal end insertable within the body of a patient and a proximal end located adjacent to the subcutaneous tissue of said body portion to be treated;
b. a cannula (40) having a least one exposed portion located at said outer surface of the body, accommodating at least a portion of the proximal end of the strip. The cannula comprises sealing means adapted to seal said cannula; and,
c. a membrane (11) interlinking or coupling at least two portions of the strip (10) in the distal end within the body.

The membrane can be flexible or rigid and composed of are and are composed of materials selected from a group consisting of metals such as stainless steel (SS), annealed 316 SS, Nitinol (NiTi), silica, polyurethane, polyamides, silk mesh, and biodegradable polymers such as PLA (polylactic acid) or PGA (polyglycolic acid).

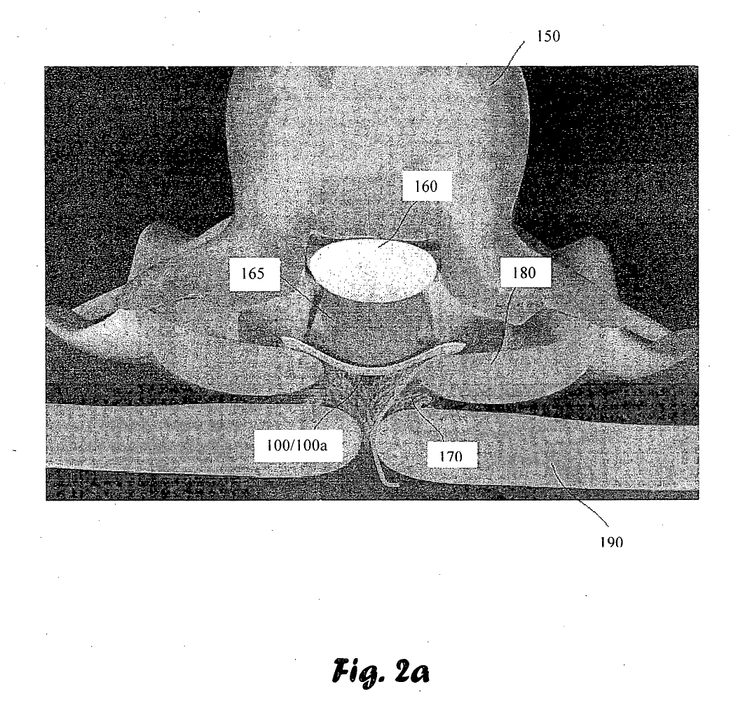

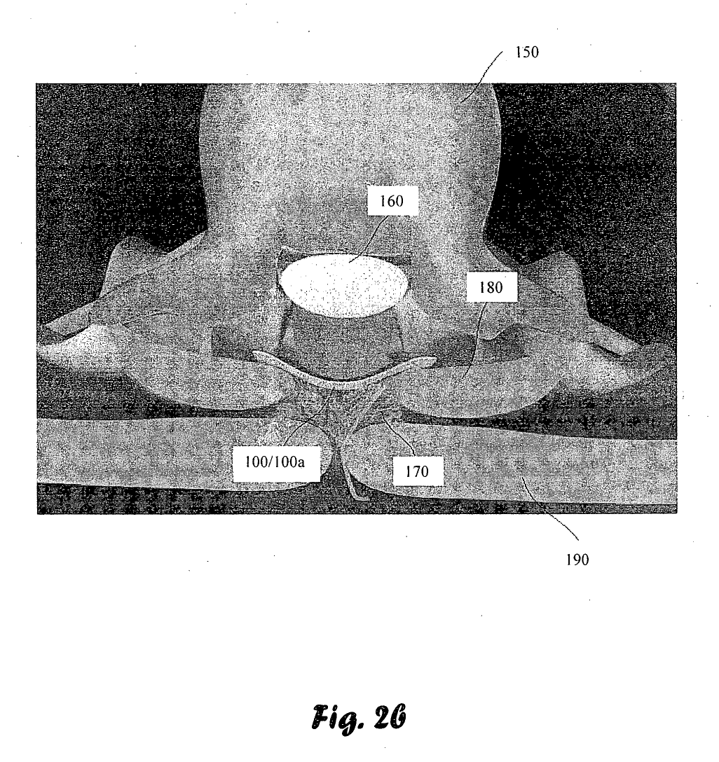

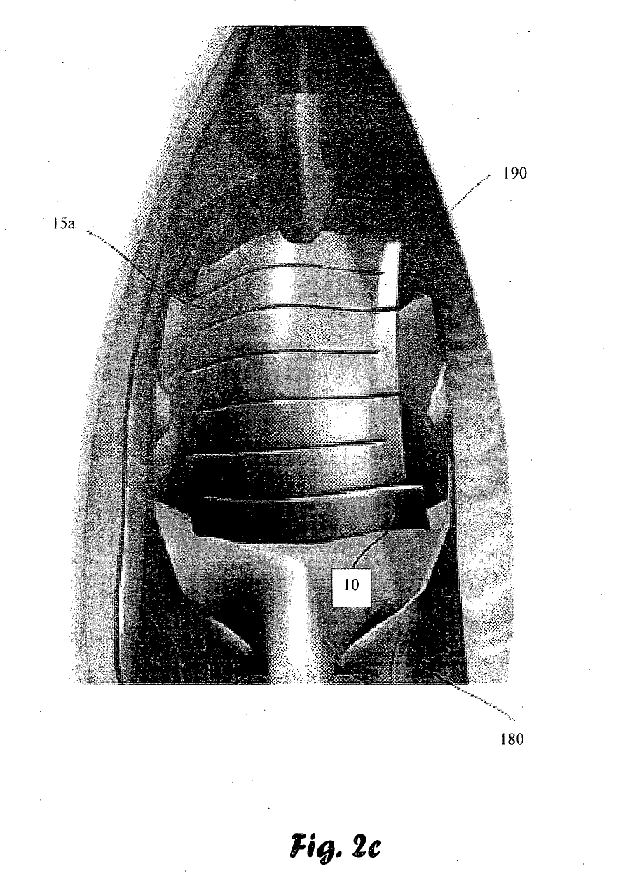

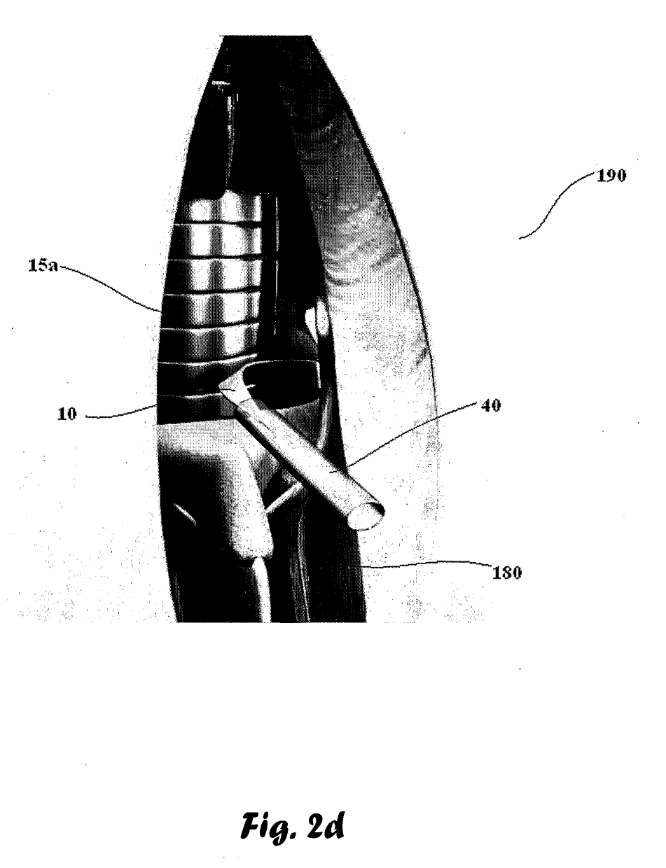

Reference is now made to FIG. 2a, showing an operative field of wide laminectomy where the lamina has been removed. A vertebra 150, a spinal cord 160, fibrosis tissues 170, subcutis 180 and dermis 190 are illustrated is FIG. 2a. It is admitted that laminectomy is likely to be accompanied by complications stemming from scarring. For preventing an implantable medical device that may be used in surgical procedures as a barrier to spatially separate an exposed tissue such as nervous tissue (for example spinal cord or nerve roots), exposed tendons and the like, from surrounding tissues, such as connecting tissue and scar tissue that may be formed at the site of injury caused by the surgical procedure, and thus prevent adherence between the exposed tissue and the surrounding tissues. The implantable medical device is adapted to be removed from the location within the patient's body at a desired time with minimal surgical intervention. The medical device may be used in such procedures as back surgeries, such as for example, lumbar laminectomy, and may include for example a lamina flavum cover that may spatially separate and thus prevent interaction between the connecting and scar tissues and the exposed nervous tissues spine after such surgery. The medical device may be removed and retrieved from the patient's body at a later time by pulling the device and unfolding the folded layered filament through a small incision in the patient's back.

Reference is now made to FIGS. 2b and 2c, showing top and back views of the laminectomy operation field with the deployed device 100a or 100. The aforesaid device 100a/100 spatially separates the spinal cord 160 and surrounding tissues 170 and parcloses a gap 165 adjacent to the spinal core 160.

Reference is now made to FIG. 2d, presenting placing the cannula 40 onto the proximal terminal of the linear member 10 and subcutaneously fixating cannula 40 at the laminectomy operation field, respectively. The proximal terminal of the linear member 10 is brought out of operation field. The cannula 40 is put on the proximal terminal of the linear member 10. Then, the cannula 40 is fixated in the subcutaneous layer 180 for a period of cicatrisation of the operation field. After a lapse of cicatrisation, the cannula 40 is denuded and the linear member 10 is pulled out without any spinal reoperation. In the figure the implantable device is positioned superficial to the bone.

It should be emphasized that the laminar member 15 is provided as a raveled structure made of the linear member 10. At the step of withdrawal, the surgeon pulls the proximal end of the linear member 10 out of cannula 40, such that the laminar member 15 unravels into the linear member 10.

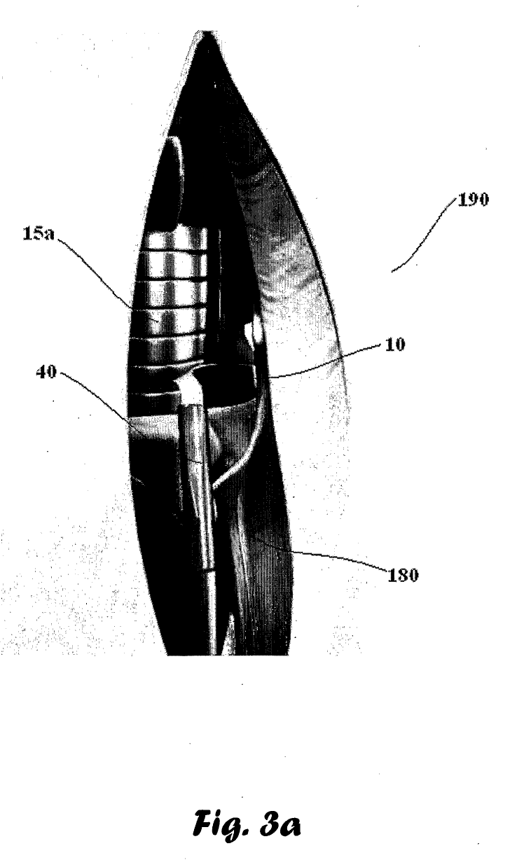

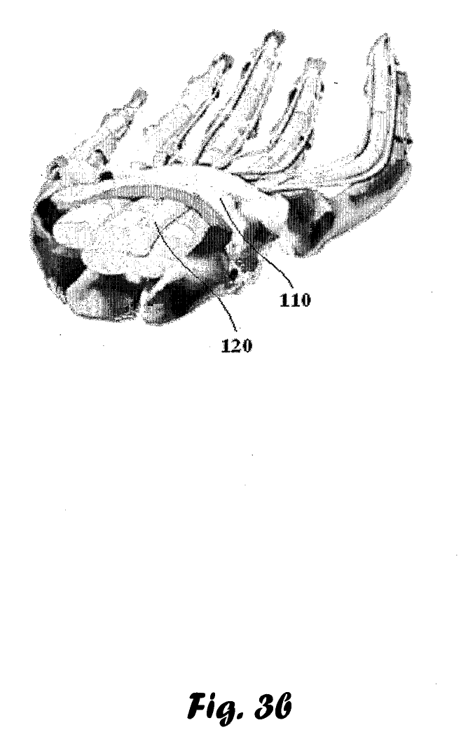

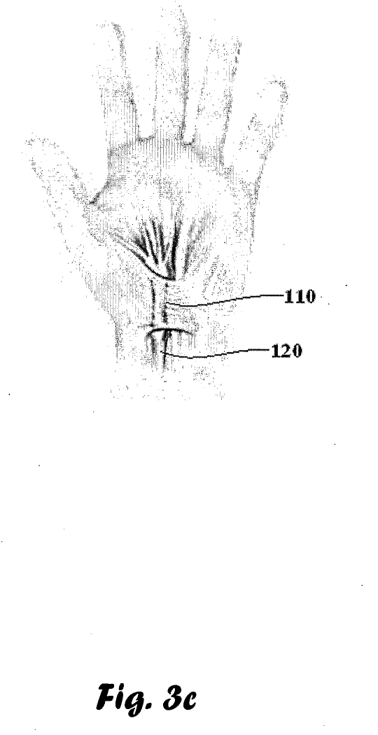

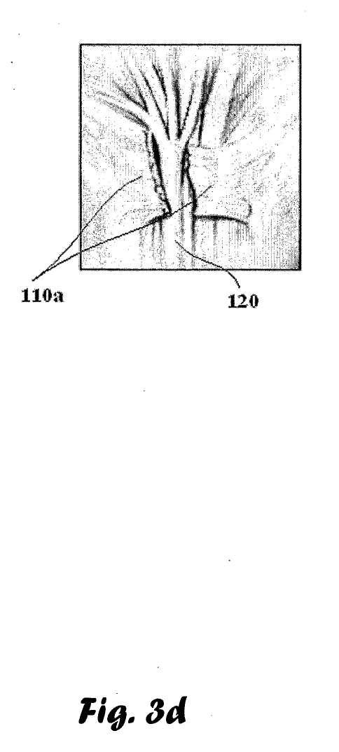

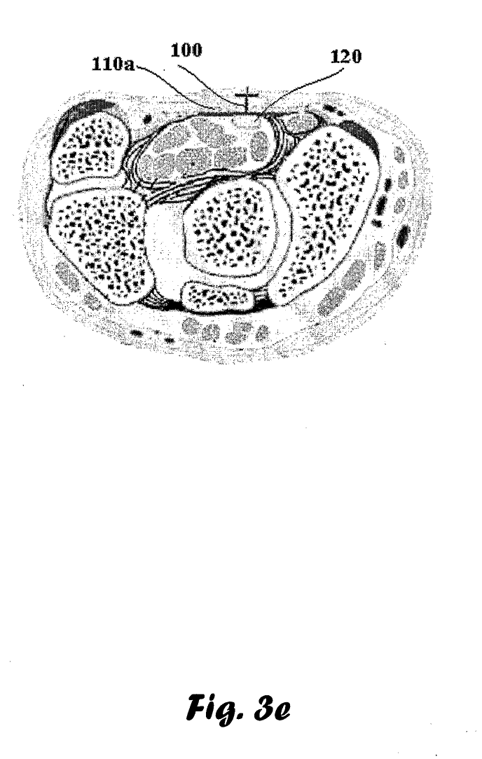

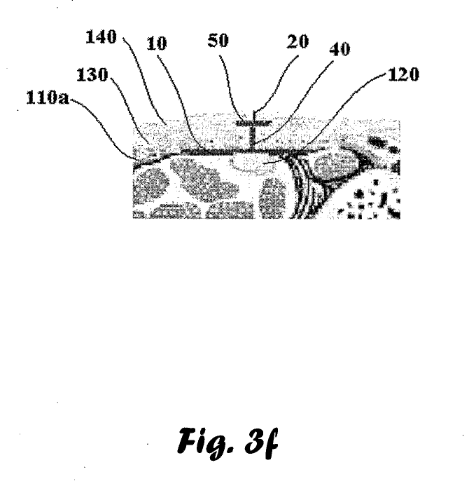

Reference is now made to FIG. 3a, presenting carpus anatomy. The median nerve 120 passes through the carpal tunnel, a canal in the wrist that is surrounded by bone on three sides, and a transverse carpal ligament 110 on the fourth. The median nerve can be compressed by a decrease in the size of the canal, an increase in the size of the contents (such as the swelling of lubrication tissue around the flexor tendons), or both.

Reference is now made to FIGS. 3b and 3c, showing pre- and post-operational views of a wrist of a patient suffering from carpal tunnel syndrome, respectively. According to FIG. 3b, the transverse carpal ligament 110 puts pressure on the median nerve 120. To release the median nerve 120 and remove pain, the surgeon divides transverse carpal ligament 110 in two ligaments 110a. Similar to laminectomy, after carpal tunnel surgical operation, there is a demand to separate the median nerve 120 and surrounding tissues.

Reference is now made to FIGS. 3d and 3e, showing the implantable medical device 100 (100a) implanted into the patient's wrist. After dividing the transverse carpal ligament 110 into 2 ligaments 110a, the surgeon specially separates the median nerve 120 and the surrounding tissues, specifically, divided ligaments 110a. The cannula 40 furnished with the plate 50 is subcutaneously fixated between the subcutis 130 and the dermis 140.

In accordance with the current invention, a temporarily implantable medical device is adapted to prevent surrounding tissues at an area of a surgical operation from mutual adhesion in a period of cicatrisation. The aforesaid medical device comprises a laminar member adapted for deploying in an area of the surgical operation and withdrawing from operative area.

The greatest innovation constitutes a laminar member comprising a linear member releasably ravelled as at least a portion of the laminar member. The laminar member is adapted to be withdrawn from the operative area by means of pulling out the linear member thereby unravelling the laminar member.

In accordance with one embodiment of the current invention, the implantable medical device is adapted to prevent the back part of a vertebra and/or nerve roots exposed after a spinal surgical operation and surrounding tissues from mutual adhesion. The medical device comprises a laminar member adapted for administration into an area of the surgical operation, deployment such that said laminar member provides spatial division of the vertebrae/nerve roots and the surrounding issues, and withdrawal from the operative area after a period of cicatrisation.

In accordance with another embodiment of the current invention, the laminar member is adapted for administration in an area of the surgical operation, and for spatial division of an object selected from the group consisting of a ligament, a tendon, a sinew, a string, and a cord from surrounding tissues. The laminar member is further adapted for withdrawal from the area after the period of cicatrisation.

In accordance with a further embodiment of the current invention, the laminar member is adapted for administration in an area of the surgical operation and the laminar member is adapted for spatial division of a peripheral nerve from the surrounding tissues. The laminar member is further adapted for withdrawal from the operative area after the period of cicatrisation.

In accordance with a further embodiment of the current invention, the laminar member comprises a linear member releasably ravelled as at least a portion of the laminar member. The laminar member is adapted to be withdrawn from the area by means of pulling out the linear member thereby unravelling the laminar member.

In accordance with a further embodiment of the current invention, the medical device is provided with a cannula at a proximal terminal of the linear member. The cannula is adapted for temporary fixation in the patient's subcutis.

In accordance with a further embodiment of the current invention, the linear member is configured into a flat-spiral-like laminar member. The laminar member is adapted for being pulled out through the cannula.

In accordance with a further embodiment of the current invention, the linear member is configured into a flat-zigzag-like laminar member. The laminar member is adapted for being pulled out through the cannula.

In accordance with a further embodiment of the current invention, the laminar member is coated with an anti-adhesive coating.

In accordance with a further embodiment of the current invention, a method of a post-operative rehabilitation after a surgical operation comprising the steps of (a) providing a temporarily implantable medical device comprising a raveled laminar member and a cannula provided at a proximal terminal of the laminar member; (b) administering the laminar member into an area of the surgical operation; (c) deploying the laminar member in the area between the objects to be divided; (d) fixating the cannula in the patient's subcutis; (e) cicatrizing the area of the surgical operation; (f) exposing the cannula; (g) withdrawing the laminar member through the cannula from the spinal surgical operation by means of unravelling the laminar member into a linear member.

In accordance with a further embodiment of the current invention, the rehabilitation is performed after a surgical operation selected from laminectomy, carpal tunnel surgery, and any operation on a ligament, a tendon, a sinew, a string, or a cord.

In accordance with a further embodiment of the current invention, the steps of administering, deploying, and withdrawing the laminar member are performed by means of a minimally invasive method.

In accordance with a further embodiment of the current invention, a reversibly implantable medical device is implanted into a predetermined body portion to be treated. The device comprises:
a. a reversibly implantable elongated continuous strip (10) of a predetermined shape (e.g., zigzag, spiral, etc.) having a biocompatible surface; said strip having a distal end within the body of a patient and a proximal end located adjacent to the subcutaneous tissue of said body portion to be treated; and,
b. a cannula (40) having a least one exposed portion located at said subcutaneous tissue of said body portion to be treated, accommodating at least a portion of said proximal end of said strip; said cannula comprising sealing means adapted to seal said cannula.

In accordance with a further embodiment of the current invention, the reversibly implantable medical device as defined above is adapted to prevent tissue adhesion, especially post-operative tissue adhesions.

In accordance with a further embodiment of the current invention, the reversibly implantable medical device as defined above is adapted for medical procedures selected from a group consisting of laminectomy, laminoplasty, scaffolding, plastic surgery, tissue reconstruction, tissue construction, sinus lifting, sinus separation, craniotomy, carpal tunnel surgery, and any operation on a ligament, a tendon, a sinew, a string, or a cord.

In accordance with a further embodiment of the current invention, the strip is adapted to form either a 2D or 3D spatial barrier.

In accordance with a further embodiment of the current invention, the shape of said barrier is selected from a group consisting of regular or irregular spirally wound, zigzag, lumen, pipe, crescent, linear, bagel-like (annular) cross section, star-like cross section, otherwise a planar or proximally planar shaped barrier of any size, shape or type, 3D configuration, and a multi-dimensional configuration comprising a plurality of sets of regular or irregular 2D planes and/or 3D spatial members interlinked together (each of said sets comprises at least two members) to form a continuous member suitable to at least partially fill the interstices between tissues and/or organs within the body.

In accordance with a further embodiment of the current invention, the tissue and/or organs are selected from a group consisting of a ligament, a tendon, a sinew, a string, peripheral nerve and surrounding tissues, a cord, and surrounding tissues thereof.

In accordance with a further embodiment of the current invention, the cannula is at least temporarily affixed to patient's subcutis and/or facia.

In accordance with a further embodiment of the current invention, the strip is coated and/or immersed and/or coupled and/or doped and/or covered and/or contained by means selected from a group consisting of anti-adhesive agents, medicaments, sustained released drugs, radio-opaque agents, bio-markers, biodegradable polymer, fillers, edema preventing members, biocides, shape memory materials, collagen enzymes, anti coagulating agents, proadhesion modifiers such as talcum powder, Si containing agents.

In accordance with a further embodiment of the current invention, the cannula has a distal end inserted into said body portion to be treated and a proximal end located adjacent to the subcutaneous tissue of said body portion to be treated; further wherein the shape of said distal end is concave and/or flared.

In accordance with a further embodiment of the current invention, the strip and/or the cannula are flexible or rigid and are composed of materials selected from a group consisting of metals such as stainless steel (SS), annealed 316 SS, Nitinol (NiTi), silica, polyurethane, polyamides, silk mesh, a biodegradable polymer such as PLA (polylactic acid) or PGA (polyglycolic acid).

In accordance with a further embodiment of the current invention, the strip has a thickness of about 50 microns to about 500 microns, preferably about 100 microns to about 200 microns; length of about 3 centimetres to about 3 meters; width of about 1 millimetre to about 3 millimetres, preferably about 1.5 millimetres.

In accordance with a further embodiment of the current invention, a method for treating a predetermined body portion is provided. The method comprises steps selected inter alia from:
a. reversibly implanting an elongated continuous strip of a predetermined shape, having a biocompatible surface, within said body portion to be treated by inserting said strip to said body portion to be treated;
b. shaping said strip within said body portion to be treated according to said predetermined shape; and,
c. providing at least a portion of said strip adjacent to the subcutaneous tissue of said body portion to be treated.

In accordance with a further embodiment of the current invention, the method as defined above, additionally comprising the final steps of:
a. reversibly locating at least a portion of a cannula at said subcutaneous tissue of said body portion to be treated and optionally at least temporarily fixating the same;
b. withdrawing said strip out of said body via said cannula.

In accordance with a further embodiment of the current invention, the method as defined above, is for preventing tissue adhesion in a predetermined body portion to be treated, especially post-operative tissue adhesions; preferably with cicatrizing tissue.

In accordance with a further embodiment of the current invention, the method as defined above, is useful for a surgical operation selected from a group consisting of laminectomy, laminoplasty, scaffolding, plastic surgery, tissue reconstruction, tissue construction, sinus lifting, sinus separation, craniotomy, carpal tunnel surgery, and any operation on a ligament, a tendon, a sinew, a string, or a cord.

In accordance with a further embodiment of the current invention, the method as defined above, additionally comprising the step of preventing tissue adhesion after a surgical operation, said operation is selected from a group consisting of laminectomy, laminoplasty, scaffolding, plastic surgery, tissue reconstruction, tissue construction, sinus lifting, sinus separation, craniotomy, carpal tunnel surgery, and any operation on a ligament, a tendon, a sinew, a string, or a cord.

In accordance with a further embodiment of the current invention, a minimally invasive method is provided.

In accordance with a further embodiment of the current invention, the method as defined above is provided, additionally comprising the step of coating and/or immersing and/or coupling and/or doping and/or covering and/or containing said strip by means selected from a group consisting of anti-adhesive agents, non-adhesive agents, medicaments, sustained released drugs, radio-opaque agents, bio-markers, biodegradable polymer, fillers, edema preventing members, biocides, anti-coagulating agents.

In accordance with a further embodiment of the current invention, a reversibly implantable medical device is provided. The device is implantable in a predetermined body portion to be treated. Said device comprises:
a. a reversibly implantable elongated continuous strip of a predetermined shape having a biocompatible surface; said strip having a distal end within the body of a patient and a proximal end located adjacent to the subcutaneous tissue of said body portion to be treated; and,
b. a cannula having a least one exposed portion located at said subcutaneous tissue of said body portion to be treated, accommodating at least a portion of said proximal end of said strip; said cannula comprising sealing means adapted to seal said cannula; and,
c. a membrane interlinking or coupling at least two portions of said strip in said distal end within said body.

In accordance with a further embodiment of the current invention, the reversibly implantable medical device as defined above is provided, wherein said membrane provides backing support for said strip.

In accordance with a further embodiment of the current invention, the device is adapted to prevent tissue adhesion, especially post-operative tissue adhesions.

In accordance with a further embodiment of the current invention, the device is especially adapted for medical procedures selected from a group consisting of laminectomy, laminoplasty, scaffolding, plastic surgery, tissue reconstruction, tissue construction, sinus lifting, sinus separation, craniotomy, carpal tunnel surgery, and any operation on a ligament, a tendon, a sinew, a string, or a cord.

In accordance with a further embodiment of the current invention, the strip is adapted to form either a 2D or 3D spatial barrier.

In accordance with a further embodiment of the current invention, the shape of said barrier is selected from a group consisting of regular or irregular spirally wound, zigzag, lumen, pipe, crescent, linear, bagel-like (annular) cross section, star-like cross section, otherwise a planar or proximally planar shaped barrier of any size, shape or type, 3D configuration, and a multi-dimensional configuration comprising a plurality of sets of regular or irregular 2D planes and/or 3D spatial members interlinked together (each set comprises at least two members) to form a continuous member suitable for at least partially filling the interstices between tissues and/or organs within the body.

In accordance with a further embodiment of the current invention, the tissue and/or organs are selected from a group consisting of a ligament, a tendon, a sinew, a string, peripheral nerve and surrounding tissues, a cord, and surrounding tissues thereof.

In accordance with a further embodiment of the current invention, the cannula is at least temporarily affixed to patient's subcutis and/or facia.

In accordance with a further embodiment of the current invention, the strip is coated and/or immersed and/or coupled and/or doped and/or covered and/or contained by means selected from a group consisting of anti-adhesive agents, medicaments, sustained released drugs, radio-opaque agents, bio-markers, biodegradable polymer, fillers, edema preventing members, biocides, shape memory materials, collagen enzymes, anti coagulating agents, proadhesion modifiers such as talcum powder, Si-containing agents.

In accordance with a further embodiment of the current invention, the cannula has a distal end inserted into said body portion to be treated and a proximal end located adjacent to the subcutaneous tissue of said body portion to be treated; further wherein the shape of said distal end is concave and/or flared.

In accordance with a further embodiment of the current invention, the strip and/or the cannula and/or the membrane are flexible or rigid and are composed of materials selected from a group consisting of metals such as stainless steel (SS), annealed 316 SS, Nitinol (NiTi), silica, polyurethane, polyamides, silk mesh, and biodegradable polymers such as PLA (polylactic acid) and PGA (polyglycolic acid).

In accordance with a further embodiment of the current invention, the membrane is produced by means of gluing or deeping, laser cutting, photoetching, moulding or thermoshaping.

In accordance with a further embodiment of the current invention, the strip having thickness of about 50 microns to about 500 microns, preferably about 100 microns to about 200 microns; length of about 3 centimetres to about 3 meters; width of about 1 millimetre to about 3 millimetres, preferably about 1.5 millimetres.

In accordance with a further embodiment of the current invention, a method for treating a predetermined body portion is provided. The method comprises steps selected inter alia from
a. providing an elongated continuous strip of a predetermined shape, having a biocompatible surface;
b. interlinking or coupling several portions of said elongated continuous strip via a membrane;
c. reversibly implanting said elongated continuous strip within said body portion to be treated by inserting said strip to said body portion to be treated;
d. shaping said strip within said body portion to be treated according to said predetermined shape; and,
e. providing at least a portion of said strip adjacent to the subcutaneous tissue of said body portion to be treated.

In accordance with a further embodiment of the current invention, the method as defined above is provided, additionally comprising the final steps of:
a. reversibly locating at least a portion of a cannula at said subcutaneous tissue of said body portion to be treated and optionally at least temporarily fixating the same;
b. withdrawing said strip out of body via said cannula.

In accordance with a further embodiment of the current invention, the method as defined above is provided, for preventing tissue adhesion in a predetermined body portion to be treated, especially post-operative tissue adhesions; preferably with cicatrizing tissue.

In accordance with a further embodiment of the current invention, the method as defined above is provided, useful for surgical operation selected from a group consisting of laminectomy, laminoplasty, scaffolding, plastic surgery, tissue reconstruction, tissue construction, sinus lifting, sinus separation, craniotomy, carpal tunnel surgery, and any operation on a ligament, a tendon, a sinew, a string, or a cord.

In accordance with a further embodiment of the current invention, the method as defined above is provided, additionally comprising step of preventing tissue adhesion after a surgical operation; said operation is selected from a group consisting of laminectomy, laminoplasty, scaffolding, plastic surgery, tissue reconstruction, tissue construction, sinus lifting, sinus separation, craniotomy, carpal tunnel surgery, any operation on a ligament, a tendon, a sinew, a string, and a cord.

In accordance with a further embodiment of the current invention, a minimally invasive method as defined above is disclosed.

In accordance with a further embodiment of the current invention, the method as defined above is provided, additionally comprising the step of coating and/or immersing and/or coupling and/or doping and/or covering and/or containing said strip by means selected from a group consisting of anti-adhesive agents, non-adhesive agents, medicaments, sustained released drugs, radio-opaque agents, bio-markers, biodegradable polymer, fillers, edema preventing members, biocides, anti coagulating agents.

It should be emphasized that the device and method as described above can be used in different medical procedures. For example the reversibly implantable medical device can be used in laminoplasty surgeries for the reconstruction of the lamina.

Furthermore, the reversibly implantable medical device, as described above, can be found useful in different plastic procedures, e.g. for tissue molding, tissue construction and re-construction, filling spaces or fragmentariness (e.g., cicatrix) in plastic surgery, etc.

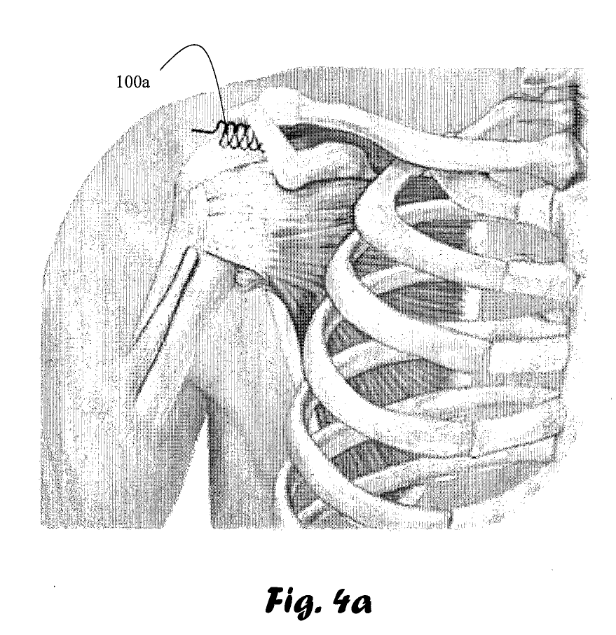

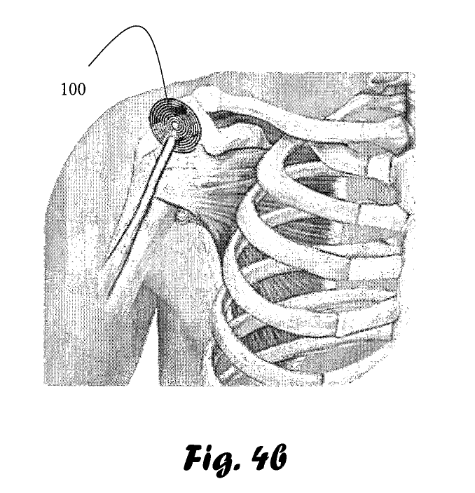

To demonstrate the different uses of the reversibly implantable medical device, reference is now made to FIGS. 4-7.

Reference is now made to FIGS. 4a and 4b which illustrate the use of the reversibly implantable medical device (according to any of the embodiments described above) in the shoulder area. FIG. 4a illustrates the use of the device shaped in a zigzag form and FIG. 4b illustrates the use of the device shaped in a spiral form.

The figures illustrate an example in which the implantable medical device is implanted, for example, in the supraspinatus rotator cuff tendons area. The device can be implanted if a portion of the rotator cuff tendons is resected or ruptured, and can be used to provide a barrier between said tendons and for example the collarbone or the scapula.

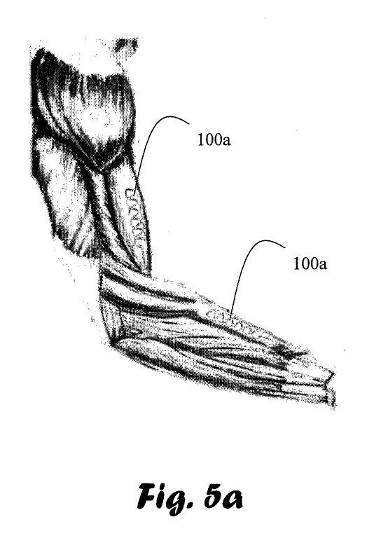

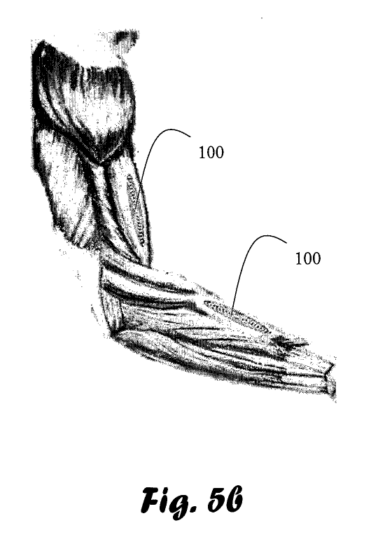

Reference is now made to FIGS. 5a and 5b which illustrate the use of the reversibly implantable medical device (according to any of the embodiments described above) in the arm area. FIG. 5a illustrates the use of the device shaped in a zigzag form and FIG. 5b illustrates the use of the device shaped in a spiral form.

The figures illustrate an example in which the implantable medical device is implanted in the biceps brachii area, the caput longum tricipitis brachii, the caput mediale tricipitis brachii, palmaris longus, the flexor Capri radialis, the flexor digitorum superficialis, the brachioradialis, the flexor pollicis longus.

The device can be implanted if a portion of each of said muscle is resected or ruptured, and can be used to provide a barrier between each of said muscle and for example the skin.

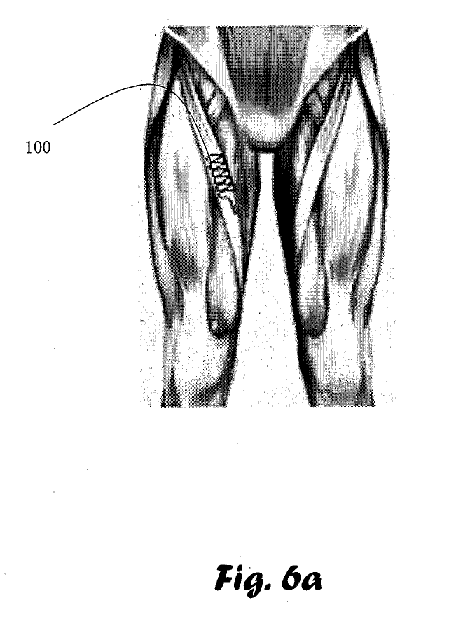

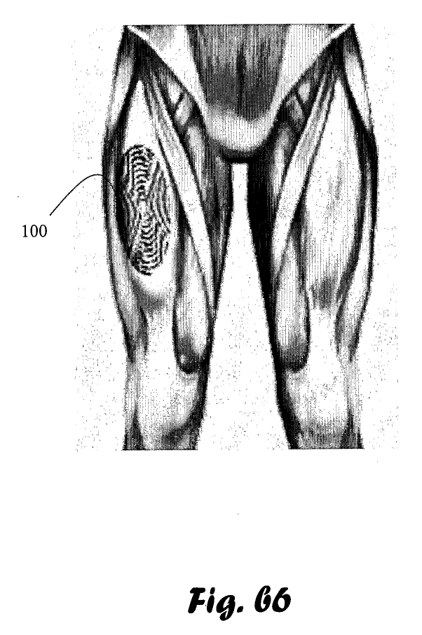

Reference is now made to FIGS. 6a and 6b which illustrate the use of the reversibly implantable medical device (according to any of the embodiments described above) in the hip or thigh area. FIG. 6a illustrates the use of the device shaped in a zigzag form and FIG. 6b illustrates the use of the device shaped in a spiral form.

The figures illustrate an example in which the implantable medical device is implanted in the sartorius muscle, tensor fascia lata muscle, rectus femoris, vastus lateralis et cetera.

The device can be implanted if a portion of each of said muscle is resected or ruptured, and can be used to provide a barrier between each of said muscle and for example the skin.

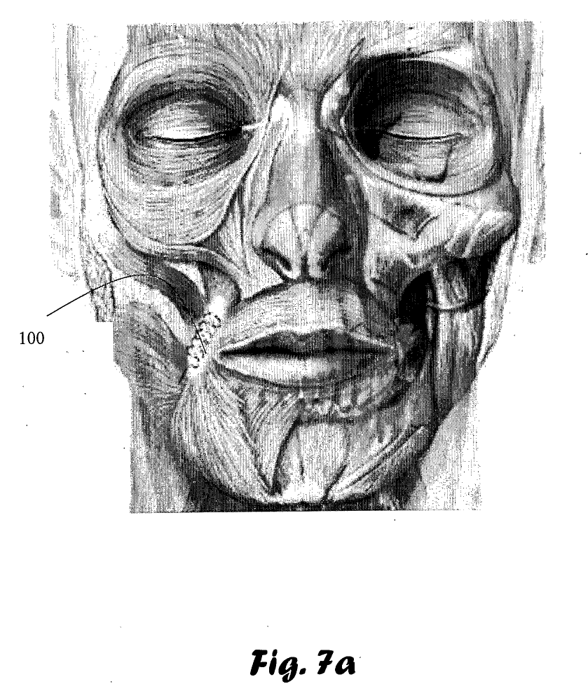

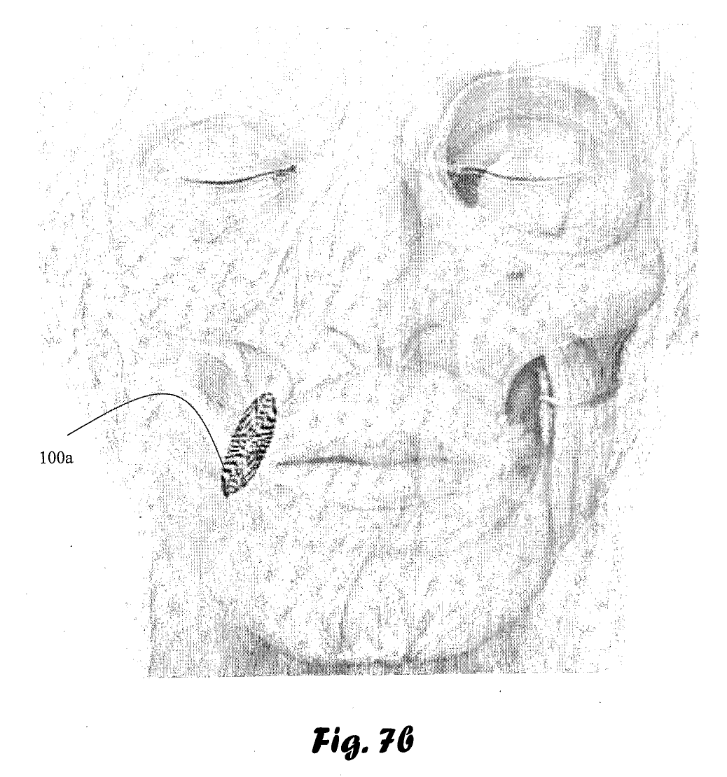

Reference is now made to FIGS. 7a and 7b which illustrate the use of the reversibly implantable medical device (according to any of the embodiments described above) in the facial area. FIG. 7a illustrates the use of the device shaped in a zigzag form and FIG. 7b illustrates the use of the device shaped in a spiral form.

These figures illustrate an example in which the implantable medical device is used in plastic surgery so as to scaffold, design and reconstruct fillers injected into a body portion to be treated. It is emphasized that the device can be used to redesign any kind of fragmentariness or/and natural cicatrix.

It should be pointed out that different fillers exist in the market and are usually administered by injection. However, the ability to control the injection, the exact positioning of the fillers and fixating the same in a specific position is still an unmet need.

In sharp contrast, the use of the device as described in the present invention meets said need since it enables exact control over the position, shape and size of the device within the body portion to be treated.

In the foregoing description, embodiments of the invention, including preferred embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principles of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A reversibly implantable medical device adapted for molding tissues; said device is implantable in a predetermined body portion to be treated; wherein said device comprises:
a. a reversibly implantable elongated continuous strip of a predetermined shape having a biocompatible surface; said strip having a distal end within the body of a patient and a proximal end located adjacent to the subcutaneous tissue of said body portion to be treated; and,
b. a cannula having a least one exposed portion located at said subcutaneous tissue of said body portion to be treated, accommodating at least a portion of said proximal end of said strip; said cannula comprising sealing means adapted to seal said cannula.

2. The implantable medical device according to claim 1, adapted to prevent tissue adhesion, especially post-operative tissue adhesions.

3. The implantable medical device according to claim 1, especially adapted for medical procedures selected from a group consisting of laminectomy, laminoplasty, scaffolding, fillers' design, plastic surgeries, tissue reconstruction, tissue construction, sinus lifting, sinus separation, craniotomy, carpal tunnel surgery, and any operation on a ligament, a tendon, a sinew, a string, or a cord.

4. The device according to claim 1, wherein said strip is adapted to form either a 2D or 3D spatial barrier.

5. The device according to claim 4, wherein the shape of said barrier is selected from a group consisting of regular or irregular spirally wound, zigzag, lumen, pipe, crescent, linear, bagel-like (annular) cross section, star-like cross section, otherwise a planar or proximally planar shaped barrier of any size, shape or type, 3D configuration, and a multi-dimensional configuration comprising a plurality of regular or irregular two or more 2D planes and/or 3D spatial members interlinked together to form a continuous member suitable to at least partially fill the interstices between tissues and/or organs within the body.

6. The device according to claim 5, wherein said tissue and/or organs are selected from a group consisting of a ligament, a tendon, a sinew, a string, peripheral nerve and surrounding tissues, a cord, and surrounding tissues thereof.

7. The implantable medical device according to claim 1, wherein said cannula is at least temporarily affixed to said patient's subcutis and/or fascia.

8. The implantable medical device according to claim 1, wherein said strip is coated and/or immersed and/or coupled and/or doped and/or covered and/or contained by means selected from a group consisting of anti-adhesive agents, medicaments, sustained released drugs, radio-opaque agents, bio-markers, biodegradable polymer, fillers, edema preventing members, biocides, shape memory materials, collagen enzymes, anti coagulating agents, proadhesion modifiers such as talcum powder, Si containing agents.

9. The implantable medical device according to claim 1, wherein said cannula has a distal end inserted into said body portion to be treated and a proximal end located adjacent to the subcutaneous tissue of said body portion to be treated and least temporarily affixed thereto; further wherein the shape of said distal end is concave and/or flared.

10. The implantable medical device according to claim 1, wherein said strip and/or cannula is composed of a material selected from the group consisting of metals such as stainless steel (SS), annealed 316 SS, Nitinol (NiTi); silica, polyurethane, polyamides, silk mesh, biodegradable polymers such as PLA (polylactic acid), PGA (polyglycolic acid) and any combination thereof.

11. The implantable medical device according to claim 1, wherein said strip having thickness of about 50 microns to about 500 microns, preferably about 100 microns to about 200 microns; length of about 3 centimetres to about 3 meters; width of about 1 millimetre to about 3 millimetres, preferably about 1.5 millimetres.

12. The implantable medical device according to claim 1, wherein said strip further comprises at least two portion; said portions are interconnected by a membrane; said membrane is adapted to provide backing support for said strip.

13. A method for treating a predetermined body portion by tissues in said body portion, comprising steps of
a. reversibly implanting an elongated continuous strip of a predetermined shape, having a biocompatible surface, within said body portion to be treated by inserting said strip to said body portion to be treated;
b. shaping said strip within said body portion to be treated according to said predetermined shape; and
c. providing at least a portion of said strip adjacent to the subcutaneous tissue of said body portion to be treated.

14. The method according to claim 13, additionally comprising the final steps of:
d. reversibly locating at least a portion of a cannula at said subcutaneous tissue of said body portion to be treated and optionally at least temporarily fixating the same; and
e. withdrawing said strip out of said body via said cannula.

15. The method according to claim 13, for preventing tissue adhesion in a predetermined body portion to be treated, especially post-operative tissue adhesions; preferably with cicatrizing tissue.

16. The method according to claim 13, useful for surgical operation selected from a group consisting of laminectomy, laminoplasty, scaffolding, filler design, plastic surgery, tissue reconstruction, tissue construction, sinus lifting, sinus separation, craniotomy, carpal tunnel surgery, and any operation on a ligament, a tendon, a sinew, a string, or a cord in a minimally invasive manner.

17. The method according to claim 13 performed in a minimally invasive manner.

18. The method according to claim 13, additionally comprising the step of coating and/or immersing and/or coupling and/or doping and/or covering and/or containing said strip by means selected from a group consisting of anti-adhesive agents, non-adhesive agents, medicaments, sustained released drugs, radio-opaque agents, bio-markers, biodegradable polymer, fillers, edema preventing members, biocides, anti-coagulating agents.

19-36. (canceled)
